(19) United States
(12) Patent Application Publication
(10) Pub. No.: US 2024/0325689 A1
(43) Pub. Date: Oct. 3, 2024

(54) SELF-GUIDING CATHETER WITH PROXIMITY SENSOR

(71) Applicant: Covidien LP, Mansfield, MA (US)

(72) Inventors: Peter D.C. INGLIS, Boulder, CO (US); Derek S. Tata, Longmont, CO (US)

(73) Assignee: Covidien LP, Mansfield, MA (US)

(21) Appl. No.: 18/600,292

(22) Filed: Mar. 8, 2024

Related U.S. Application Data
(60) Provisional application No. 63/492,866, filed on Mar. 29, 2023.

(51) Int. Cl. A61M 25/01

(52) U.S. Cl. CPC A61M 25/0105; A61M 2025/0166; A61M 2205/3306; A61M 2205/3327; A61M 2205/3368; A61M 2205/3375; A61M 2205/3561; A61M 2205/507; A61M 2205/52; A61M 2210/1035; A61M 2210/1039

(57) ABSTRACT

The present technology relates to a catheter navigation system that includes a catheter comprising a steerable distal tip including a proximity sensor. The system also includes a catheter controller. The catheter controller includes a processor; and memory storing instructions that, when executed by the processor, cause the catheter controller to perform operations. The operations may include receiving, from the catheter, a proximity signal generated by the proximity sensor, identifying a steering target based on the proximity signal, and generating a steering signal to steer the steerable distal tip towards the steering target.

### CROSS-REFERENCE TO RELATED APPLICATIONS

This application claims the benefit of U.S. Provisional Application No. 63/492,866 filed Mar. 29, 2023, titled “Self-Guiding Catheter with Proximity Sensor,” which is incorporated herein by reference in its entirety.

### BACKGROUND

Catheters, introducers, and endoscopes are generally long, flexible instruments that can be introduced into a cavity or lumen of a patient during a medical procedure in a variety of situations to facilitate visualization and/or medical procedures within the cavity. Such medical instruments may be inserted into a patient's mouth, throat, trachea, esophagus, or into other cavities such as blood vessels. The medical instrument, such as the catheter, may include a steerable distal tip that can be actively controlled to bend or turn the distal tip in a desired direction to navigate through the anatomy.

The position and arrangement of airway passages or other cavities or lumens is variable between patients. Thus, to assist in navigation of catheters, endoscopes, or introducers, a model or estimation of the patient anatomy can be created prior to a procedure for an individual patient using external imaging modalities, such as computer tomography (CT) or magnetic resonance imaging (MRI).

### SUMMARY

Certain embodiments commensurate in scope with the originally claimed subject matter are summarized below. These embodiments are not intended to limit the scope of the disclosure. Indeed, the present disclosure may encompass a variety of forms that may be similar to or different from the embodiments set forth below.

In an aspect, the technology relates to a catheter navigation system that includes a catheter comprising a steerable distal tip including a proximity sensor; and a catheter controller, coupled to the catheter. The catheter controller includes a processor; and memory storing instructions that, when executed by the processor, cause the catheter controller to perform operations. The operations include receiving, from the catheter, a proximity signal generated by the proximity sensor; identifying a steering target based on the proximity signal; and generating a steering signal to steer the steerable distal tip towards the steering target.

In an example, the proximity sensor includes at least one of an infrared sensor, an acoustic sensor, or a temperature sensor. In another example, the proximity sensor includes at least one proximity receiver positioned at least partially around a circumference of the distal tip. In still another example, identifying the steering signal is further based on an anatomical model generated by an external imaging modality. In yet another example, the steerable distal tip further includes a position sensor, and generating the steering signal is further based on orientation data received from the position sensor. In still yet another example, identifying the steering target comprises providing the proximity signal as input into a trained machine-learning (ML) model; and receiving, as output from the ML model, the steering target. In a further example, the steering target is identified by identifying features of the proximity signal characteristic of a lumen and selecting a center of the lumen as the steering target.

In another aspect, the technology relates to a catheter navigation system that includes a catheter comprising a steerable distal tip including a proximity sensor; and a catheter controller, coupled to the catheter. The catheter controller includes a processor; and memory storing instructions that, when executed by the processor, cause the catheter controller to perform operations. The operations include receiving, from the catheter, a proximity signal generated by the proximity sensor; based on the proximity signal, determining a proximity of the steerable distal tip to a lumen wall of a lumen; and based on the determined of the steerable distal tip to the lumen wall, generating a steering signal to steer the steerable distal tip away from the lumen wall and towards a center of the lumen.

In an example, determining the proximity of the steerable distal tip to one or more lumen walls includes determining a first distance of a first side of the distal tip to a first lumen wall; and determining a second distance of a second side of the distal tip to a second lumen wall, wherein the second distance is greater than the first distance; and wherein the steering signal is to steer the distal tip towards the second side of the distal tip. In another example, the operations further include, based on the determined proximity, identifying the center of the lumen. In yet another example, the steering signal is a first steering signal, and the operations further include based on the proximity signal, identifying a bifurcation of a first lumen and a second lumen of a patient; based on an anatomy model of the patient, identifying a first lumen as providing a pathway to a navigation target in the anatomy model; and generating a second steering signal to steer the distal tip into the first lumen. In still another example, the proximity sensor includes a proximity emitter; and at least one at least one proximity receiver positioned at least partially around a circumference of the distal tip.

In another aspect, the technology relates to a method for navigating a catheter while the catheter is being advanced through a lumen. The method includes receiving, at a catheter controller, a proximity signal from a proximity sensor in a steerable distal tip of the catheter, the proximity sensor being at least one of an infrared sensor, an acoustic sensor, or a temperature sensor; providing the proximity signal to a trained machine-learning (ML) model; receiving an output form the ML model in response to the proximity signal; identifying a steering target based on the output from the ML model; and automatically bending, by the catheter controller, the distal tip towards the steering target.

In an example, the steering target is a central axis of the lumen. In another example, the lumen is a first lumen, the catheter is positioned at a bifurcation to a second lumen and third lumen, and the steering target is one of the second lumen or the third lumen.

In another aspect, the technology relates to a catheter navigation system that includes a catheter comprising a steerable distal tip including a proximity sensor; and a catheter controller, coupled to the catheter. The catheter controller includes a processor; and memory storing instructions that, when executed by the processor, cause the catheter controller to perform operations. The operations include receiving, from the catheter, a proximity signal generated by the proximity sensor; based on the proximity signal, detecting an anatomical structure in proximity to the distal tip; and generating a steering signal to steer the steerable distal tip away from the detected anatomical structure. In an example, the anatomical structure is a lumen wall. In another example, the anatomical structure is tissue dividing two lumens at a junction.

Features in one aspect or embodiment may be applied as features in any other aspect or embodiment, in any appropriate combination. For example, features of a system, handle, controller, processor, scope, method, or component may be implemented in one or more other system, handle, controller, processor, scope, method, or component.

### BRIEF DESCRIPTION OF THE DRAWINGS

Advantages of the disclosed techniques may become apparent upon reading the following detailed description and upon reference to the drawings in which:

FIG. 1 depicts a view of a catheter navigation system including a catheter with a proximity sensor.

FIGS. 2A-2F depict example proximity sensor configurations at the distal tip of the catheter.

FIG. 3 depicts a navigation system with the catheter in a first position.

FIG. 4 depicts a navigation system with the catheter in a second position.

FIGS. 5A-5B depict a schematic illustration of automatic steering of a catheter that tracks alignment to a center of a passage.

FIG. 6 is a view showing locations of proximity data acquired during steering within the patient anatomy.

FIG. 7 is a schematic block diagram of a navigation system.

FIG. 8 depicts an example method for automatically steering a catheter based on proximity data.

FIG. 9 depicts another example method for automatically steering a catheter based on proximity data.

### DETAILED DESCRIPTION

Catheters, endoscopes, and introducers are thin, elongated, flexible instruments that can be inserted into a body cavity for exploration, imaging, biopsy, or other clinical treatments. While the below description primarily refers to catheters, as used herein, catheters are generally intended to include other types of elongate medical instruments, such as endoscopes, introducers or the like. The catheter may be formed as a tube made of various materials such as silicone, rubber, or plastic, and can range in diameter from a few millimeters to several centimeters, depending on its intended use. Catheters often feature a hollow center that allows for the passage of fluids or gases, and they can be used for a variety of medical purposes, such as injecting contrast dye into blood vessels or removing fluid from the lungs.

Catheters may be navigated into the body cavity (such as a patient's airway, gastrointestinal tract, oral or nasal cavity, or other cavities or openings) via advancement of the distal tip to a desired position and, in certain embodiments, via active steering of the distal tip of the catheter. Catheters may also be inserted through the skin into a vessel or other lumen of the body in percutaneous catheterization procedures.

Advancement of catheters into patient cavities is typically via force transferred from a proximal portion of the device (outside of the patient cavity) that results in advancement of the distal tip within the patient cavity. As used herein, “proximal” refers to the direction out of the patient cavity, back toward the handle end of a device, and “distal” refers to the direction forward into the patient cavity, away from the doctor or caregiver, toward the probe or tip end of the device. For example, a doctor or other caregiver holding a proximal portion of the catheter outside of the patient cavity pushes downward or forward, and the resulting motion is transferred to the distal tip of the catheter, causing the tip to move forward (distally) within the cavity. Similarly, a pulling force applied by the caregiver at the proximal portion may result in retreat of the distal tip or movement in an opposing (proximal) direction out of the patient cavity. The catheter, or catheter controller, may also include steering controls to change orientation at the distal tip based on operator input to navigate or point the catheter in a desired direction.

Because patient cavities are not regularly shaped or sized, the catheterization procedure may include navigation through an unpredictable and tortuous path to reach a particular point in the anatomy (such as reaching into branches of the lungs). While some catheters, such as endoscopes, may have a camera at the distal tip to provide images as the endoscope is advanced, many other catheters do not have cameras. Such catheters are inserted into the body without exact knowledge of their position unless additional live or real-time external images sources (e.g., x-ray devices, fluoroscopes, MRI, etc.) are used to track the location of the catheter. Required use of external imaging devices increases the need for additional large-scale equipment, and the continued use of ionizing radiation, is generally undesirable.

Even in examples where the catheter includes a distal camera (e.g., an endoscope), acquiring images in some cavities may not be possible (e.g., smaller vessels) and the image alone fails to indicate the absolute position of the distal tip in the body. Moreover, as the catheter passes through the body, bodily fluids such as mucus, blood, or other tissues may gather on the distal tip, which occludes the view of a camera.

Among other things, the technology of the present application addresses the above limitations by incorporating a proximity sensor at the distal tip of the catheter. Some examples of proximity sensors may include an infrared sensor, an acoustic sensor (e.g., ultrasonic sensor), and a thermal sensor, among others. The proximity sensor may be incorporated in addition or alternatively to a camera depending on the example. The proximity sensor is able to sense the structures of lumen or cavity (e.g., lumen walls, bifurcations) into which the distal end of the catheter is inserted. Steering of the catheter may then be based on tracking the proximity of the distal tip of the catheter relative to the lumen structures. For instance, based on the proximity of the distal tip to a lumen wall, the distal tip may be automatically steered away from the wall and more towards the center of the lumen. By avoiding the walls of the lumen, the potential for injury to the patient may be avoided and a smoother insertion process may be achieved.

In addition to steering of the catheter, the proximity data may also be used to determine the position of the distal tip within the anatomy of a patient. For example, a patient-specific model (e.g., generated from external imaging) or a generic patient model representative of patient anatomy (which may be selected from an available set of models based on patient demographic, size, age, gender, etc.) may be used to help locate the catheter within the patient. As the catheter progresses through the patient, the identification of junctions or intersections of lumens may be identified based on the proximity data. The position corresponding to the junction may then be identified in the anatomy model, and the position of the distal tip of the catheter may be correlated with the corresponding position in the anatomy model. Such identification may be performed in real time, and position indicators of the catheter may be generated and presented during the procedure. In addition, the anatomy model may provide additional navigation information on which the automatic steering of the catheter may be based.

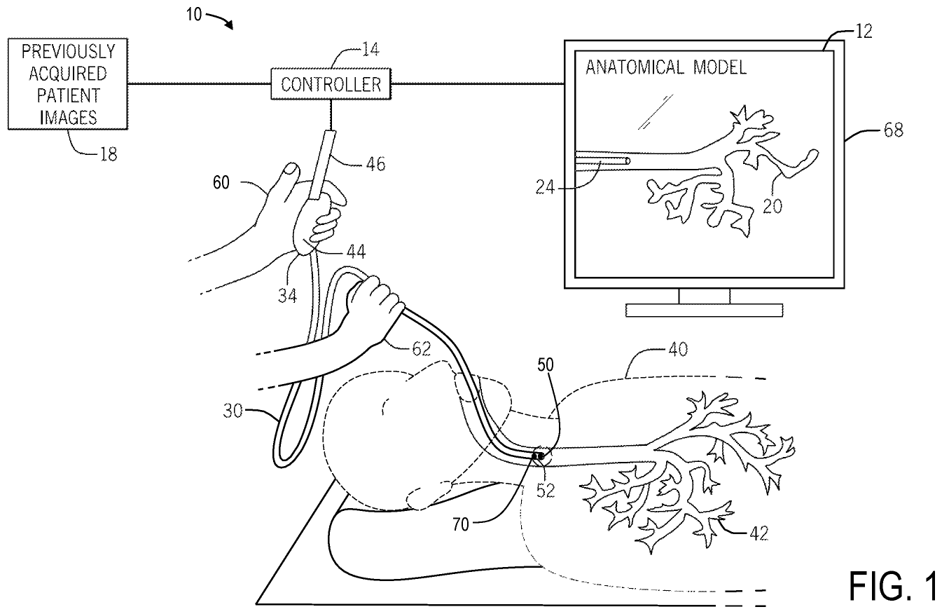

FIG. 1 depicts a view of an elongate-instrument navigation system 10. The elongate-instrument navigation system 10 is described below with respect to an example where the elongate instrument is a catheter, but it should be understood that the system 10 could be used with other types of elongate instruments.

The navigation system 10 includes a catheter 30 that is inserted into the body of a patient 40. The catheter 30 may be manually advanced into the patient 40 by a clinician. The catheter 30 includes a distal tip 52 at the distal end of the catheter 30 (e.g., the end of the catheter 30 that is inserted into the patient 40). The distal tip 52 includes a proximity sensor 50 and may also include other types of sensors, such as a position sensor 70 (e.g., inertial measurement unit (IMU)). The distal tip 52 may be steerable via bending or articulating. Steering of the distal tip 52 may be controlled via various steering systems that cause the distal tip 52 to bend in a desired direction. As one example, the bending the distal tip 52 may be controlled via pull wires that run the length of the catheter 30.

The bending of the distal tip 52 is controlled via a catheter controller 34. The proximal end of the catheter 30 is connected to the catheter controller 34. Steering signals generated by the catheter controller 34 may then be used by the steering actuators for the catheter 30 to cause the distal tip 52 to bend in a direction and magnitude indicated by the steering signals. In some examples where the catheter 30 is being inserted into the airways of the patient 40, the catheter controller 34 may be a video laryngoscope.

In some examples, the clinician who is operating the catheter 30, holds a handle 44 of the catheter controller 34 with his or her left hand 60, and grips or pinches the catheter 30 with his or her right hand 62. The operator can move the catheter 30 proximally or distally with the right hand 62.

The catheter controller 34 may also include a display screen 46 that displays images. The images may be images captured from a camera of the catheter (where one is present) and/or a camera of the video laryngoscope in examples where the catheter controller 34 is a laryngoscope. In an embodiment, the display screen 46 is a touch screen, and the operator can input touch inputs on the screen 46 (such as with the operator's left thumb) to steer the distal end of the catheter 30, such as to bend it right, left, up, or down. As such, both manual steering of the distal tip 52 (e.g., based on clinician inputs) and automatic steering of the distal tip 52 are possible with the present technology. While the catheter 30 is being automatically steered, an automatic steering indicator may also be displayed on the on the display screen to indicate to the operator that automatic steering in ongoing. In some examples, the automatic steering may only occur when an automatic steering mode of the catheter controller 34 is activated or selected.

The example navigation system 10 may also include an anatomy model 12. The anatomy model 12 may be created in various different ways, such as using previously acquired images 18 or anatomical data from the patient. In an embodiment, the model 12 is created from an external imaging scan of the patient prior to the catheterization procedure. The scan can be CT (computed tomography), MRI (magnetic resonance imaging), x-ray, or other diagnostic or imaging scans. These scans can be used to build a three-dimensional model of the actual anatomy of an individual patient. For example, computations from a CT scan can be used to build a three-dimensional model of a patient's airways (for example, computer-based methods for segmentation of anatomy based on CT scans). The resulting three-dimensional model shows the actual unique airway branches of that individual patient, as the airways split and branch out below the left and right bronchial tubes.

In FIG. 1, the anatomy of the model 12 is the patient's airways, but it should be understood that other anatomy models can be used in other procedures and contexts, including for example models of the vascular structures, skeletal features, soft tissue, and/or gastrointestinal structures, among others. The anatomy model 12 may be displayed as a simplified model generated from rich image data. That is, the overall display of the anatomy model 12 may be a cartoon view, line drawing, three-dimensional model, or section view of the airways. The anatomy model 12 may be rendered to show the approximate locations and dimensions of lumens (e.g., airway passages 42) and surrounding tissue walls, such as the tracheal or bronchial walls. For example, using CT scan data including density information, the anatomy model 12 can be generated based on density rules to designate less dense areas as likely to be open airway passages and more dense areas as likely to be tissue. Airway walls of the anatomy model are rendered based on tissue areas located at the border of open airway passages. In such an example, the tissue walls can be designated in the anatomy model 12 with or without fine feature resolution or texturing. The anatomy model 12 can be generated by or an updated by the system 10 as provided herein.

The anatomy model 12 may be displayed on a display screen 68 as computer-generated graphics 20 during a catheter procedure. The computer-generated graphics 20 may also include a simulated catheter 24 tracking progress of the real-world catheter 30 in real time. The simulated catheter 24 is a computer-generated animation that represents the actual catheter 30 that is being moved within the patient 40.

The simulated catheter 24 tracks real-world movement of the catheter 30 caused by operator manipulation in a distal or proximal direction and/or orientation changes mediated by operator steering inputs to the catheter controller 34. The graphics controller 14 renders the simulated catheter 24 within the anatomy model 12 and moves the simulated catheter 24 within the model 12 in coordination with movements of the real-world catheter 30. The position of the simulated catheter 24 within the anatomy model 12 represents the actual position, which may include an orientation or pose, of the real-world catheter 30 within the patient 40. Thus, when the operator advances the catheter 30 distally within the patient, the graphics controller 14 adjusts the rendering of the simulated catheter 24 to move the simulated catheter 24 a corresponding distance through the model 12. The simulated the simulated catheter 24 24 may be a marker showing the live, real-time, moving position of the catheter 30 within the global map of the model 12.

The real-time location of the catheter 30 is provided based on position data from position sensor 70 and/or proximity data captured by the proximity sensor 50 located at the distal tip 52. The proximity sensor 50 and the position sensor 70 capture live signals during a catheter procedure that are provided in substantially real time to the graphics controller 14 via communication between the catheter 30 and the controller 14 (which may be via the catheter controller 34). In some examples, the functions of the graphics controller 14 are incorporated into the catheter controller 34.

As the real-world position of the distal tip 52 changes, the position sensor 70 generates updated position signals based on the movement of the distal tip 52. The proximity sensor 50 also generates proximity data based on the relative location of the distal tip 52 to the anatomical structures (e.g., lumen walls). The proximity data and/or the position data may then be used to steer the distal tip 52 of the catheter 30 and/or update the position of the simulated catheter 24 in the model 12. In some examples where the catheter also includes a camera, the steering the of the distal tip 52 and/or the rendering of the simulated catheter 24 may also be based on images captured by the camera, as discussed in U.S. application Ser. No. 18/050,013, titled Endoscope with Automatic Steering, and U.S. application Ser. No. 17/719,007, titled Endoscope Navigation System with Updating Anatomy Model, both of which are incorporated by reference in their entireties. To the extent the discussion in the present application conflicts with either of those references, the discussion of the present application shall control.

The anatomy model 12, however, may not fully align with the live patient anatomy. In some instances, such inaccuracies may result in the real-world anatomy (e.g., airway passages 42) being slightly shifted, stretched, or changed relative to the corresponding locations in the model 12. Even a model 12 that is initially correctly aligned to the patient 40 may become less accurate over the course of a clinical procedure due to patient movement, position shifts, or changes in health status. Further, the generated model 12 may include inherent inaccuracies in size, scale, or presence/absence of anatomical features based on the resolution limits of the imaging technology or patient-specific variables that influence image quality. In another example, inaccuracies in the anatomy model 12 may be based on patient position differences used in scanning versus the catheter procedure. For example, CT images used to generate the anatomy model 12 may be acquired with the patient's arms positioned above their head and with the patient holding in a full breath. In contrast, patients undergoing catheter procedures may generally be arranged with their arms by their sides and breathing independently or via intubation and mechanical ventilation. Such differences in patient positioning and breathing may cause associated position shifts in the airway passages 42, rendering the anatomy model 12 at least partly inaccurate. While these inaccuracies may be on a millimeter scale and may only be within discrete regions of the model 12, such differences may result in steering difficulties when the operator is using the anatomy model 12 to navigate.

As provided herein, the navigation system 10 incorporates real-time signals from the proximity sensor 50 and/or the position sensor 70 to update the anatomy model 12 and/or the position of the simulated catheter 24 within the model. These real-time signals may be used in conjunction with the anatomy model 12 to generate steering signals for the distal tip of the catheter 30. For instance, based on the proximity data, a junction or bifurcation of two lumens may be identified. The corresponding bifurcation in the anatomy model 12 may then be identified. Based on an ultimate target or navigation plan, the distal tip 52 of the catheter 30 may be automatically steered into the correct or desired lumen.

In some examples, the real-time proximity signals and/or position signals may also be used to update and/or confirm portions of the anatomy model 12. The updating may include correcting portions of the anatomy model 12, adjusting a scale of the anatomy model 12, changing relationships between features of the anatomy model 12 (such as stretching or compressing portions of the model), adjusting orientation of structures in the model, and/or adjusting the rendering of the simulated catheter 24 within the anatomy model 12 by way of example. For instance, the proximity data may indicate a diameter of a lumen, and such data may be used to adjust or update the anatomy model 12.

The updating may also include rerouting of suggested navigation routes from the simulated catheter 24 to a desired navigation target based on the updated anatomy model 12. In an embodiment, the rendering of the simulated catheter 24 is updated in the anatomy model 12 without changing the dimensions or characteristics of the model itself. For example, if the anatomy model 12 differs from the live patient physiology in a particular way (such that the actual catheter 30 has reached a particular feature in the patient, but the simulated catheter 24 has not reached the corresponding feature in the model 12), the simulated catheter 24 may be moved within the anatomy model 12 to correspond to the position of the catheter 30 in the patient (such as by moving the simulated catheter 24 within the anatomy model 12 to the feature). In this way, provide accurate navigation to the user, the simulated catheter 24 can be moved to track the live catheter 30 without actually changing the model 12 itself. Thus, in an example, updating the model includes updating the placement, orientation, or movement of the simulated catheter 24 within the anatomy model 12.

As the catheter 30 advances through the airway passages 42, the acquired proximity data from the proximity sensor 50 and/or position data from the position sensor 70 are used to steer the distal tip 52 of the catheter 30 and/or update the anatomy model 12. Accordingly, in some examples, a catheter 30 may be advanced into the patient 40 and be automatically routed or steered to an ultimate destination within the patient 40. Such steering may not only include maintaining the distal tip 52 within a central portion of the lumen (e.g., to avoid contacting the lumen walls), but the steering may also include navigating through junctions or bifurcations to reach a desired target within the patient 40. As such, a clinician may only need to continue to advance the catheter 30 into the patient 40, and the automatic steering of the present technology guides the catheter 30 to the navigation target with little to no input from the clinician.

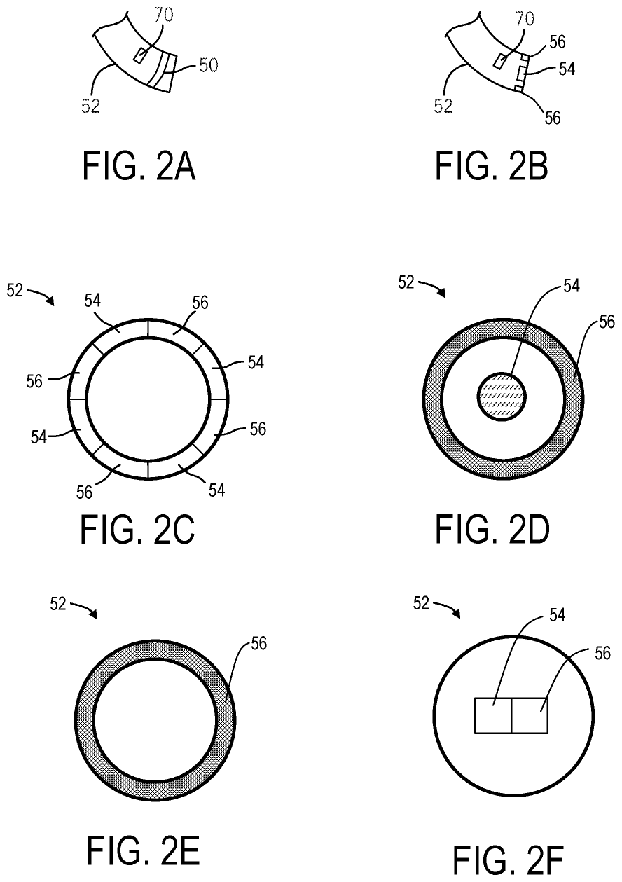

FIGS. 2A-2F depict example proximity sensor configurations at the distal tip 52 of the catheter. The proximity sensor 50 may be positioned at various positions at the distal tip 52 that provide differing sensing capabilities. In some examples, the proximity sensor 50 may be positioned at the terminus of the distal tip 52, which allows for light or acoustic signals to be emitted from the proximity sensor in a predominantly distal direction. In other examples, the proximity sensor 50 may be positioned offset from the terminus of the distal tip 52 and may be positioned around (or partially around) the distal tip 52. The proximity sensor 50 and the position sensor 70 may be mounted on the same circuit board (e.g., flex circuit) and electrically connected to the catheter controller via wires that extend through the length of the catheter.

FIG. 2A depicts side view of an example catheter where the proximity sensor 50 is positioned around, or at least partially around, the circumference of the distal tip 52. By positing the proximity sensor 50 around the circumference, the proximity sensor 50 is able to more directly sense the proximity of an outer wall or surface of the distal tip 52 to the anatomical wall or structure of the lumen. While the proximity sensor 50 is positioned around the circumference, the proximity sensor 50 does not have to be on the exterior surface of the distal tip 52. Rather, the proximity sensor 50 may be below (or interior to) the exterior surface of the distal tip 52, and the proximity sensor 50 is distributed radially around a central axis (extending in a proximal-to-distal direction) of the distal tip 52.

In some examples, the proximity sensor 50 is comprised of a proximity emitter 54 and a proximity receiver 56. The emitter 54 emits light signals and/or acoustic signals that are then detected by the receiver 56 after the light signals and/or the acoustic signals are reflected from (or otherwise affected by) the anatomy of the patient (e.g., lumen walls, junction structures). For instance, the receiver 56 may be a transducer converts physical energy of the environment around the receiver 56 to an electric signal that can be processed and analyzed as discussed herein.

For example, where the proximity sensor 50 is an infrared sensor, the emitter 54 is a light emitter (e.g., light-emitting diode) that emits light in the infrared or near-infrared spectrum. In such infrared examples, the receiver 56 is an infrared light-receiver, which may be in the form an infrared image detector and/or one or more photodiodes, photosensors, etc. that are configured to detect light in the infrared band at which the light emitter 54 emits the infrared light. When using infrared light, the specific frequency band of infrared light that is emitted may also be selected based on the particular application. For instance, when the catheter procedure is within a blood vessel and the distal tip 52 is surrounded by blood, some frequencies may be less likely to be absorbed by the blood itself. In other examples where the catheter is inserted into the airways and the distal tip 52 is likely to be covered in mucus, some infrared frequencies may penetrate the mucus better than others. Accordingly, the infrared frequencies that are utilized may be selected to produce photons that: (1) penetrate the fluid or material that is likely to cover the distal tip 52; and (2) reflect from the anatomical walls of interest (e.g., lumen walls).

In some examples, the particular frequency of light that is emitted may be selected or adjusted via the catheter controller. For instance, a particular application or anatomy may be selected that causes the corresponding frequency of light to be selected and emitted from the emitter 54. This may be accomplished by using multiple LEDs that each have a corresponding frequency band. When a particular frequency band is selected, the corresponding LED is activated or used to generate the light. In other examples, the light frequency of the individual catheter may not be adjusted. Rather, multiple catheters may be available and tuned for different anatomy or applications. For instance, each different catheter may have a different LED that emits light in a different frequency band.

The infrared sensor 50 may utilize structured light patterns in some examples to determine depth changes around, or in front of, the distal tip 52. Alternatively or additionally, time-of-flight (ToF) may be utilized to determine depths or distances from the distal tip 52 to the anatomical structures. Stereo vision techniques may also be used in some examples.

As another example, the proximity sensor 50 may be an acoustic sensor (e.g., an ultrasonic sensor). In some examples, the acoustic sensor includes an emitter 54 and a receiver 56. For instance, the acoustic emitter 54 emits acoustic pulses and the acoustic receiver 56 detects the acoustic pulses that reflect from the anatomical structures. The time of flight of the acoustic waves from being emitted to being detected then may be used to determine distances of the distal tip to different anatomical structures, such as lumen walls or junction structures.

In other examples, the acoustic sensor may only include acoustic receivers 56 and no acoustic emitters 54. In such examples, the rate of fluid (e.g., air, blood) flowing past the acoustic receiver 56 may be detected by the acoustic receiver (which may effectively be a pressure sensor). Because fluid flow has different properties near a lumen wall as compared to the center of the lumen, the rate of flow of the fluid is indicative of proximity to the lumen wall. Accordingly, the use of one or more acoustic receivers may be used to determine proximity of the distal tip 52 to a lumen wall. The actual rate of fluid (e.g., a distance per time measure) need not be determined. Rather, a relative value measured by receiver 56 that is correlated with the fluid flow may be measured and retained. The relative differences in that value may then be used to estimate proximity.

In another example, the proximity sensor 50 may be a temperature or thermal sensor. In such examples, the proximity sensor 50 may include only proximity receivers 56 rather than proximity emitters 54. For example, multiple temperature sensors (e.g., thermistors, thermocouples, thermopiles, etc.) may be distributed radially around the distal tip 52. The temperature sensors then measure the temperature at the respective positions of the temperature sensors. The temperature may have a relationship to proximity to a lumen wall. For instance, the temperature may be higher near a lumen wall than in the center of the lumen. Accordingly, if the temperature detected by a first temperature sensor is higher than the temperature detected by second temperature sensor, the distal tip 52 may not be positioned within the center of the anatomical lumen (e.g., airway, vessel). A steering signal may then be generated to turn the distal tip in the direction of the second temperature sensor (i.e., the temperature sensor with the lower temperature measurement).

FIG. 2B depicts side view of an example catheter where a proximity emitter 54 is positioned near the central axis of the distal tip 52 and proximity receivers 56 are positioned further away from the central axis than the proximity emitter. In such examples, the proximity emitter 54 is able to emit signals that travel distally into the lumen, and the receivers 56 are able to resolve spatial reflections of the emitted signals in a manner that allows for generating a steering signal in the direction of one or more of the receivers.

FIGS. 2C-2F depict cross sections of the distal tip 52 along a plane that is perpendicular to a central axis of the distal tip 52. FIG. 2C depicts an example distal tip 52 where the emitters 54 and the receivers 56 are distributed around the circumference (or distributed radially) of the distal tip 52.

FIG. 2D depicts an example distal tip 52 with an emitter 54 near the central axis of the distal tip 52 and a receiver 56 around the circumference of the distal tip 52. The receiver 56 may be a plurality of discrete receivers and/or otherwise configured to determine spatial signal strength. For instance, the receiver 56 may be configured to determine the signal strength of the reflected signal at different radial positions, which may correspond to discrete pixels or acoustic transducer elements.

FIG. 2E depicts an example distal tip 52 that includes a radially distributed receiver 56 but does not include a proximity receiver 54. Like the receiver in FIG. 2D, the receiver 56 may be a plurality of discrete receivers and/or otherwise configured to determine spatial signal strength.

FIG. 2F depicts an example distal tip 52 that includes an emitter 54 and a receiver 56 near central axis of the distal tip 52. In such examples, the emitter 54 may emit a cone-shaped beam of light or acoustic waves that are reflected from the anatomical structures around of and/or in in front of the distal tip 52. The receiver 56 detects the reflected signals and tracks where the signals are reflected from to assign coordinates or positional signal strength. For instance, the receiver 56 may assign signal strength to different pixels that within the field of view of the receiver. Based on the different signal strengths of the pixels, a steering signal may be generated to steer the distal tip 52.

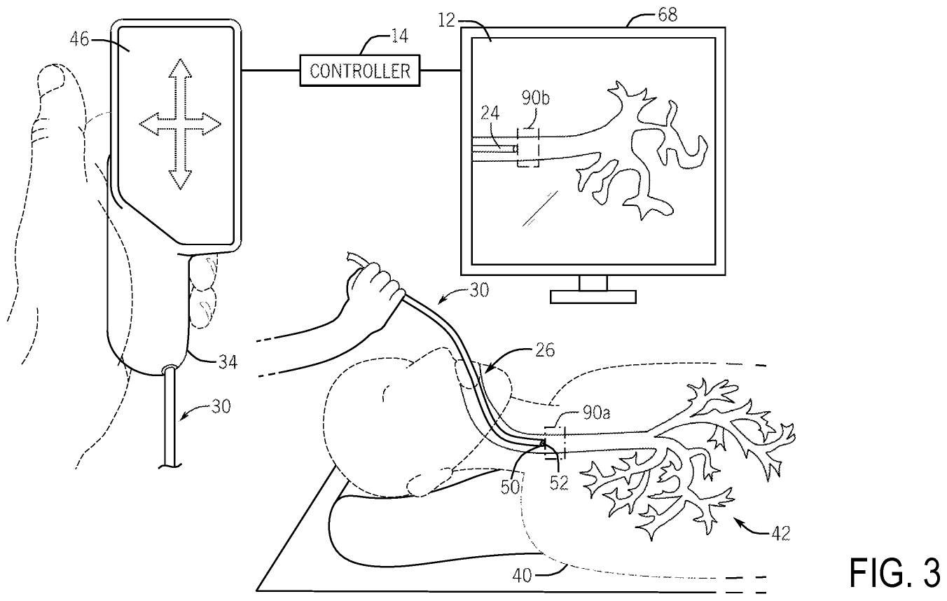

FIG. 3 is a system view including an example live view of the anatomical model 12 with a simulated catheter 24 representing a position of the real catheter 30. In FIG. 3, the real catheter 30 is positioned at a first region 90a in the patient anatomy that corresponds to a region 90b in the model 12.

The rendering of the simulated catheter 24 within the model 12 may be based on the stream of live proximity data and/or position data and updated in real time. For example, the stream of live data may be used to correct any relative position inaccuracies and to account for patient position shifts over the course of the procedure. This correction to the model 12 can be done without relying on patient position sensors within the room. The model 12 is updated and synchronized to the actual live proximity data coming from the catheter 30, regardless of how the patient is oriented in the room. Rather than using an active external patient sensing device, such as an electromagnetic sensing pad underneath the patient 40, patient movement can be accounted for based on the synchronization between the patient 40 and the model 12.

In an embodiment, the system 10 may perform an initial registration of the anatomy model 12 with the patient 40 by synchronizing the location of the simulated catheter 24 and real-world catheter 30, such as by registering the simulated catheter 24 at the lips of the model 12 when the real-world catheter 30 passes the patient's lips. In an example, the anatomy model 12 is registered at least in part relative to one or more detectable exterior patient features, such as a detected nasal opening, lips, or shoulder of the patient. The initial registration provides a starting point for rendering the simulated catheter 24 within the model 12 according to the actual position of the catheter 30 within the patient 40.

When the distal tip 52 of the catheter is in the first region 90a, the lumen continues in a contiguous manner (e.g., does not branch) according to the model 12. Accordingly, based on the model 112, the steering instruction for the distal tip may be to continue moving along the central axis of the lumen. The determination that the lumen does not branch at the first region 90a may also be based on, or confirmed by, proximity data from the proximity sensor 50. For instance, the proximity sensor 50 may indicate that for some distance distal to the distal tip 52 is not occluded by other anatomical structures that would indicate a branch or junction of lumens.

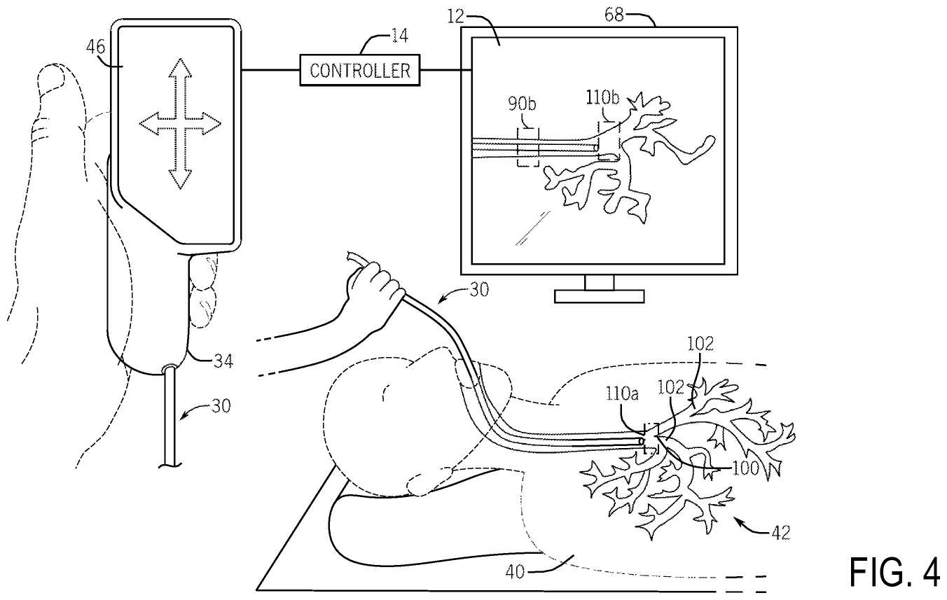

In FIG. 4, the catheter has been advanced distally, moving the catheter 30 further forward into the patient airway to a second region 110a, toward a junction or branching of the lumens. In the example depicted, the junction is the carina 100, which is the tissue at the end of the trachea, where the trachea divides into the left and right bronchial tubes 102L, 102R. As shown in the anatomy model 12 in FIG. 4, the simulated catheter 24 has moved forward distally within the anatomy model 12, toward the carina 100, and is represented as being in corresponding region 110b of the model 12.

The updated position of the distal tip 52 may be determined based on the proximity data of the proximity sensor 50 and/or the position data from the position sensor 70. For instance, the forward/distal movement of the distal tip 52 may be measured or estimated based on measurements from the position sensor 70 (e.g., accelerometer). When the anatomy model 12 may then indicate that a junction should be present at current location. The detection of the junction or branch may also or alternatively be detected from the proximity data from the proximity sensor 50. For instance, the pattern of a bifurcation or junction may be identified from the proximity data. The anatomy model 12 may then be used to determine which lumen of the bifurcation should be followed by the catheter 30.

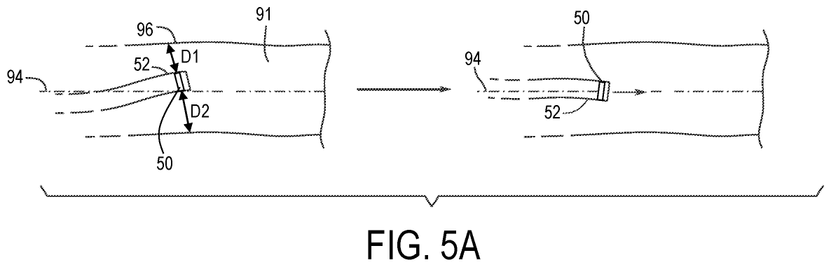

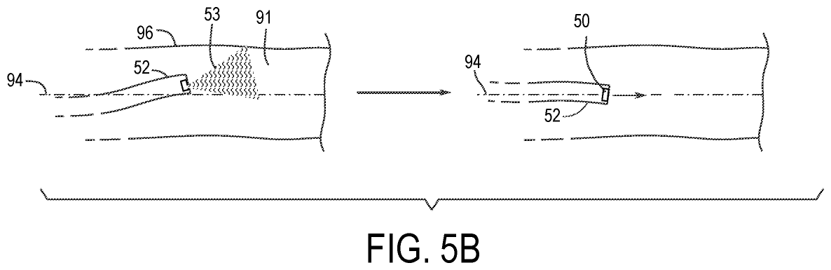

FIGS. 5A-5B depict a schematic illustration of automatic steering of a distal tip 52 catheter that tracks alignment to a center of a lumen 91. FIG. 5A shows a corresponding change in the orientation of the distal tip 52 of the catheter within the lumen 91 as a result of the automatic steering of the distal tip. Starting from the left side of FIG. 5A, the distal tip 32 is not oriented toward the target (a point on the central axis 94 of the lumen 91). Instead, in this example, the distal end 52 is oriented towards the walls 96 of the lumen 91 rather than being straight or generally oriented toward the center (e.g., towards a point along a central axis 94). In this orientation, further distal movement could cause the distal tip 52 to collide with the walls 96 of the passage, which could impede further distal movement and/or cause injury to the patient. For example, this undesired orientation of the distal end 52 may be caused by the operator inadvertently oversteering, by the operator intentionally pausing distal movement and steering the camera to view the walls 96 or some other portion of the anatomy, or because of a natural curve of the lumen 91.

When the distal tip 52 is in this position, the proximity data from the proximity sensor 50 indicates that the distal tip 52 is not aligned with the central axis 94 of the lumen. For instance, the proximity sensor 50 may indicate that one side of the distal tip 52 is closer to the lumen wall 96 than another side of the distal tip 52. As an example, the distance D1, between a first side of the distal tip 52 and the lumen wall 96, may be less than a distance D2 between a second side of the distal tip 52 and the lumen wall 96. An automatic steering signal or instruction may then be generated to steer the distal tip 52 back to towards the central axis 94. For example, the automatic steering signal steers the distal tip 52 in a direction towards the side that has the greatest distance from the lumen wall 96.

As another example, using the proximity data as an input, a feature identification model identifies the lumen 91 (e.g., via identification of the walls 96 and/or identification of a negative space indicative of the lumen 91) and, in an embodiment, the catheter controller may estimate a location of a center of the lumen 91. The steering controller then generates steering instructions to point the distal end 52 towards the center of the lumen 91. Execution of the steering instructions causes the distal end 52 to bend, move, or rotate toward the center. After executing these instructions, the distal end 52 is generally oriented along the center axis 94 and pointed towards a location corresponding to the center of the lumen 91.

FIG. 5B similarly depicts a corresponding change in the orientation of the distal tip 52 of the catheter within the lumen 91 as a result of the automatic steering of the distal tip 52. Starting on the left-hand side, the distal tip 52 is again not pointed along the central axis 94. In this example, an emitter of the proximity sensor 50 emits light or acoustic signals 53 distally from the distal tip 52. The reflected light or acoustic signals 53 are then detected by a receiver of the proximity sensor 50. Based the received signals 53 form the basis for the proximity data in such examples. The proximity data may then be used to determine whether the distal tip 52 is directed towards the target and/or to steer the distal tip 52 towards the target.

The analysis of the proximity data may be performed through a series or rules or heuristics and/or through the use of trained models. For example, a portion of the detected reflected signal that has the weakest signal (e.g., amplitude) may correspond to the center of the lumen 91 because the center of the lumen 91 is least likely to reflect the light or acoustic signals. Accordingly, steering instructions may be generated to steer the distal tip 52 towards the area of the proximity data in having the weakest or lowest amplitude. Determination of the weakest or lowest amplitude may be an average amplitude of a set of positions or pixels within the proximity data. For instance, a set of N number of adjacent pixels or positions having the lowest average amplitude may be considered the center of the lumen 91 and therefore the steering target. Gradients of the amplitudes may also be used to determine steering targets within the proximity data. For instance, the use of gradients may be useful for two-dimensional planes with relatively noisy data. Alternatively or additionally, proximity data may be provided into a feature identification model that identifies the center the lumen 91.

The feature identification model may be a machine learning (ML) or artificial intelligence (AI) model that is trained using a supervised, semi-supervised, or unsupervised manner. In an embodiment, the feature identification model may be trained using a set of proximity data and associated labels that identify a steering target and/or the center of the lumen 91. The labeling of the training data may be performed manually to generate the training data. This training data, with the associated labels, is then used to train the ML model. The feature identification model may use Haar cascades, Histogram of Gradients with Support Vector Machines (HOG+SVM), or a deep-learning model (e.g., convolutional neural network), among other types of ML models. A best-performing model that most accurately identifies and correctly labels lumens in the set of proximity data may be selected.

The feature identification may be two-step process to identify the actual steering target of the feature. For instance, a first model may identify a particular feature (e.g., a lumen). The particular steering target within the feature may then be identified or selected through the use of a second model or a set a rules/heuristics. For instance, the identified feature may be a lumen, and the steering target may be the center of that lumen. As another example, multiple lumens may be identified at a bifurcation, and the steering target may be the center of one of those lumens.

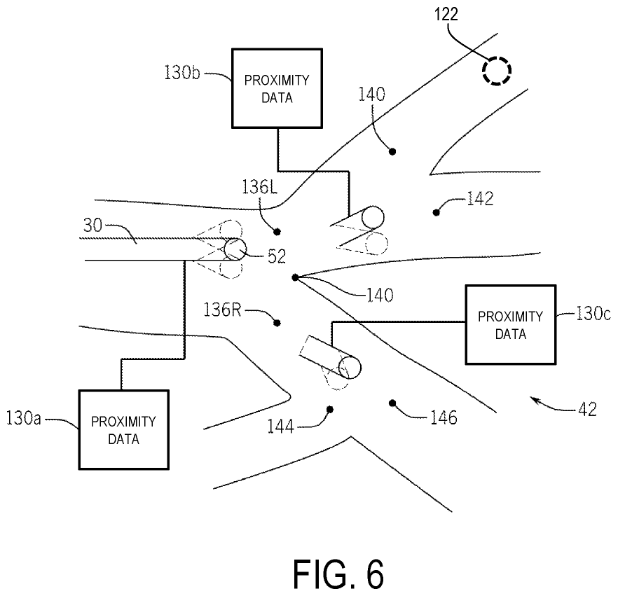

In addition to automatically steering the catheter to stay centered within a lumen, the present technology may also assist in steering the distal tip 52 into a particular lumen in a multi-lumen branch. FIG. 6 shows an example of catheter navigation based on landmark recognition from proximity data 130 and/or position data. Landmark recognition may involve processing the proximity data to identify features that are present in the proximity data 130 such as the vocal cords, bifurcations/junctions of lumens, cardiac features, or other anatomy landmarks For example, proximity data 130a may be acquired when the catheter is at position in which a left bronchial opening 136L and a right bronchial opening 136R are positioned distally from the distal tip 52, but the proximity data 130a still captures the presence of such bronchial openings. The bronchial openings, and the carina 140, can be identified from rules-based processing of the proximity data 103a. For example, the carina 140 is located at a proximal-most bifurcation of the bronchial tree distal of the vocal cords. Based on registration of the distal tip 52 with the anatomy model 12, the positional data may be used to determine how far the distal tip 52 has advanced into the body. Based on that estimate and the measurements of the anatomy model, a prediction of when the carina 140 will be encountered may be made. For instance, the carina 140 is likely to be located at the first bifurcation point distal of the vocal cords. The proximity data of the bifurcation include generally include two low-amplitude regions (indicating large depth from the distal tip 52) separated by a high-amplitude region (indicating shallower depth from the distal tip 52) that joins the low-amplitude regions. One or more ML models may also be used to process the proximity data to identify the bifurcations or other features.

Openings or bifurcations 140, 142, 144, 146 within the left and right bronchial passages can follow similar rules-based identification based on low-amplitude regions separated by a higher-amplitude region in the proximity data. Lumen openings may be designated by low-amplitude areas having a higher-amplitude perimeter. For instance, when the distal tip 52 is in the left bronchial opening 136 L, second proximity data 130b may be acquired and processed to identify the lumens 140, 142. Similarly, when the distal tip 52 is in the right bronchial opening 136R, third proximity data 130c may be acquired and processed to identify lumens 144, 146.

In some examples, the anatomy model may include a navigation target 122, which may be an anatomical point of interest, such as polyp or a surgical site. Through the use of the anatomy model, navigation to the target 122 may be more easily achieved. For instance, when each bifurcation is reached, a clinician viewing the updating model may provide a steering input to steer the distal tip 52 through the correct lumens that lead to the navigation target 122. In other examples, when each bifurcation is reached, the steering system may automatically steer the distal tip 52 into the correct lumen leading to the navigation target 122. For instance, in the example depicted, when the distal tip 52 reaches the first bifurcation, the distal tip 52 is automatically steered into lumen 136L. When the distal tip 52 then reaches the second bifurcation, the distal tip is steered into the lumen 140.

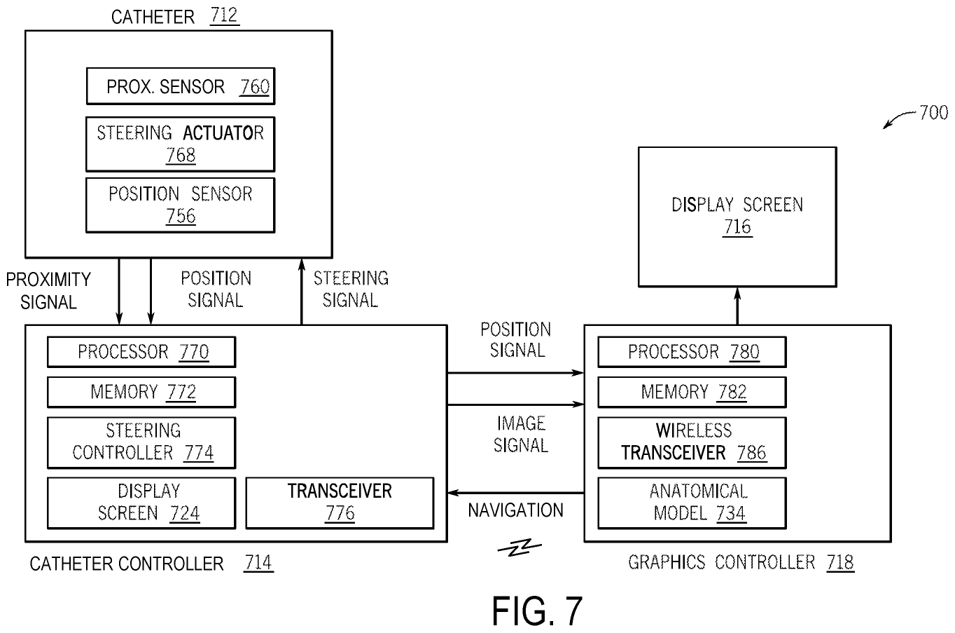

A block diagram of a navigation system 700 is shown in FIG. 7. As shown, the system includes a catheter 712, a catheter controller 714, a graphics controller 718, and separate display 716. The catheter 712 includes a proximity sensor 760 (which may include one or more emitters and/or receivers), a steering actuator 768 (coupled to the distal steerable tip of the catheter to bend or un-bend the distal tip), and a position sensor 756.

The catheter 712 is connected by a wired (shown) or wireless connection to the catheter controller 714, which includes a processor 770, hardware memory 772, steering controller 774 (such as a motor or other driver for operating the actuator 768), display screen 724, and wireless transceiver 776. The catheter controller 714 is connected by a wired or wireless (shown) connection to the graphics controller 718, which also includes a processor 780, hardware memory 782, wireless transceiver 786, and the stored anatomy model 734. The graphics controller 718 may be, for example, a laptop or desktop computer running software stored on the memory 782. The graphics controller 718 is connected by a wired or wireless (shown) connection to the display 716. In an embodiment, the display 716 is a hardware display screen fixedly or portably mounted in an environment of the patient. In one embodiment, the display 716 may be an augmented reality viewer such as goggles or glasses that includes hardware components that display the anatomy model 734 overlaid on the patient.

In an example, the catheter 712 includes one, two, or more steerable segments the form the distal tip. Each steerable segment can articulate independently of the other segments. Each steerable segment may bend and curve in three dimensions (not just in a single plane, such as up/down or right/left), curving to points in all directions up to a limit of its range of motion. For example, in an embodiment each segment can bend up to 90 degrees in any direction, enabling it to move within a hemisphere having a radius equal to the segment's length. Each segment is manipulated by its own actuation system, including one or more actuators (such as sleeved pull-wires or other actuators described below), which moves to bend or un-bend the segment into or out of a curved or bent shape.

Each articulating segment at the distal end of the catheter is manipulated by a steering system (such as steering controller 774), which operates an actuator (such as steering actuator 768) that is coupled to the segment to bend or straighten the segment. The steering system may include one or more memory metal components (e.g., memory wire, Nitinol wire) that changes shape based on electrical input, a piezoelectric actuators (such as the SQUIGGLE motor from New Scale Technologies, Victor NY), a retractable sheath (retractable to release a pre-formed curved component such as spring steel which regains its curved shape when released from the sheath), mechanical control wires (pull wires), hydraulic actuators, servo motors, or other means for bending, rotating, or turning the distal end or components at the distal end of the catheter.

The block diagram of FIG. 7 also shows the signal flow between the various devices. In an embodiment, the catheter 712 sends a live proximity signal (from the proximity sensor 760) and a live position signal (from the position sensor 756) to the catheter controller 714. The catheter controller 714 may also forward the proximity signal and the position signal to the controller 718, such as through the wireless transceivers on the two devices, and/or through wired connections and/or intermediary devices.

The controller 718 receives the proximity signal and position signal and uses that information to adjust the anatomy model and the rendering of the simulated catheter in the anatomy model. The controller 718 may also generate navigation data that may be provided to the catheter controller 714. The steering controller 774 may use the navigate data to steer the catheter towards a target identified in the anatomical model 734. For instance, at a bifurcation or junction, the navigation data may be used to steer the catheter 712 into a particular lumen that leads towards a target.

The position sensor 756 is an electronic component that senses the position and orientation (such as orientation relative to gravity) and/or movement (acceleration) of the distal tip of the catheter. The position sensor 756 contains a sensor or a combination of sensors to accomplish this, such as accelerometers, magnetometers, and gyroscopes. The position sensor 756 may generate absolute position data of the catheter distal tip or position data relative to a fixed reference point. The position sensor 756 may be an inertial measurement unit (IMU). The position sensor 756 detects static orientation and dynamic movement of the distal tip of the catheter and provides a signal indicating a change in the catheter's orientation and/or a motion of the catheter. The position sensor 756 sends this signal to the catheter controller 714. The position sensor 756 is located inside the tubular housing of the catheter 712. As shown in FIGS. 1-2, in an embodiment, the position sensor 756 is located very close to the terminus of the distal tip of the catheter to enable the position sensor 756 to capture much of the full range of movement of the distal tip and proximity sensor 760. In an embodiment, the position sensor 756 is placed at a distal end of the first steerable portion, remote from the proximal end of the steerable portion, to place the position sensor 756 away from the fulcrum of movement.

In an embodiment, the position sensor 756 generates a position signal with position coordinates and heading of the distal tip of the catheter 712. The controller 718 uses this coordinate and heading information to adjust the anatomy model 734 and the simulated catheter 24. For example, when the real-world catheter 712 is moved distally by a distance of 1 mm inside the patient, this change in position is reported by the position sensor 756 through the position signal. The new position coordinates are received by the controller 718, and the simulated catheter 24 is moved forward (distally) by the same or proportional amount within the anatomy model 734. The new position is then rendered graphically in the display 716. The data signal from the position sensor 756 may be referred to as an orientation signal, movement signal, or position signal.

The processors (such as 770, 780) may be a chip, a processing chip, a processing board, a chipset, a microprocessor, or similar devices. The controllers 714, 718 and the display 716 may also include a user input (touch screen, buttons, switches). The controllers 714, 718 may also include a power source (e.g., an integral or removable battery) that provides power to one or more components of the controller, catheter, or viewer as well as communications circuitry to facilitate wired or wireless communication with other devices. In one embodiment, the communications circuitry may include a transceiver that facilitates handshake communications with remote medical devices or full-screen monitors. The communications circuitry may provide the received images to additional monitors in real time.

The processor may include one or more application specific integrated circuits (ASICs), one or more general purpose processors, one or more controllers, FPGA, GPU, TPU, one or more programmable circuits, or any combination thereof. For example, the processor may also include or refer to control circuitry for the display screen. The memory may include volatile memory, such as random access memory (RAM), and/or non-volatile memory, such as read-only memory (ROM).

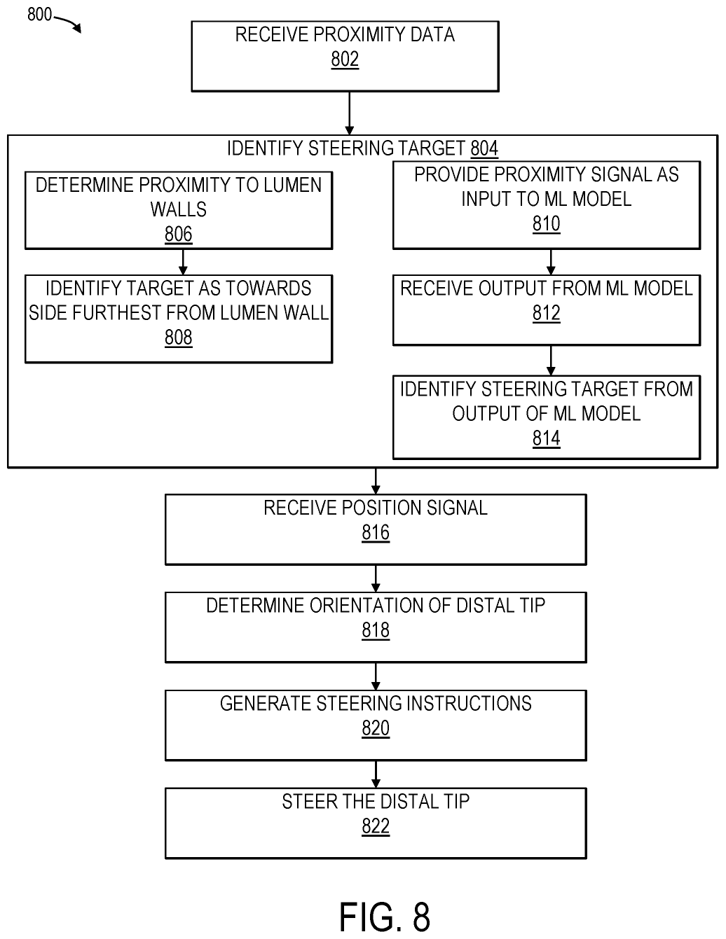

FIG. 8 depicts an example method 800 for automatically steering a catheter based on proximity data. At operation 802, proximity data is received by a proximity sensor positioned in the distal tip of the catheter. The proximity sensor may include at least one of an infrared sensor, an acoustic sensor, and/or a temperature sensor.

At operation 804, a steering target is identified. The steering target may be a center or central axis of a lumen or another lumen in a bifurcation among other possible anatomical targets. The identification of the steering target may be performed in different manners depending on the embodiment. For instance, in some examples, operations 806 and 808 may be performed. In other examples, operations 810-814 may be performed. In yet other examples, combinations of operations 806-814 may be performed. In some examples, the identifying the steering target may also or alternatively include identifying an anatomical structure (e.g., lumen wall, tissue between lumens at a junction) based on the proximity data. The steering target and/or steering signal is then to steer away from the detected anatomical structure.

At operation 806, a proximity of the distal tip to the lumen walls is determined. Such a determination may include determining the distance from the multiple sides of the distal tip to the closest proximal wall to the respective side of the distal tip. For instance, the determined distance or proximity to the walls may be measured along a distance that is substantially orthogonal to the exterior surface of the distal tip. As an example, operation 806 may include determining a first distance of a first side of the distal tip to a first lumen wall (e.g., a first portion of the lumen wall that is closest to the first side) and determining a second distance of a second side of the distal tip to a second lumen wall (e.g., a portion of the lumen wall that is closest to the second side).

At operation 808, a steering target is determining as being towards the side that is furthest from the lumen wall. For example, if all sides of the distal tip are equidistant from the lumen wall, the distal tip is likely centered within the lumen, which may be the goal or target of automatically navigating or steering the catheter. Accordingly, by steering the catheter towards the side that is furthest from the lumen wall, the distal tip will begin turning towards the center the of the lumen.

At operation 810, the proximity signal is provided as input into a trained ML model. The ML model may be a feature identification model that identifies features within the proximity data. For example, the feature identification model identifies a anatomical feature in the proximity data, such as a lumen. The feature identification model may identify the features by identifying patterns within the proximity data. For example, a low-amplitude portion of the proximity data surrounded by a high-amplitude portion may be indicative of a lumen with the low-amplitude portion corresponding to the center of the lumen. At operation 812, an output from the ML model is received in response to the input of the proximity signal. In some examples, the output includes an identification of the steering target. In other examples, the output includes only an identification of one or more features, such as a lumen. At operation 814, a steering target is identified based on the output of the ML model. Where one lumen is identified as a feature, a center or approximate center of the lumen may be identified as the steering target.

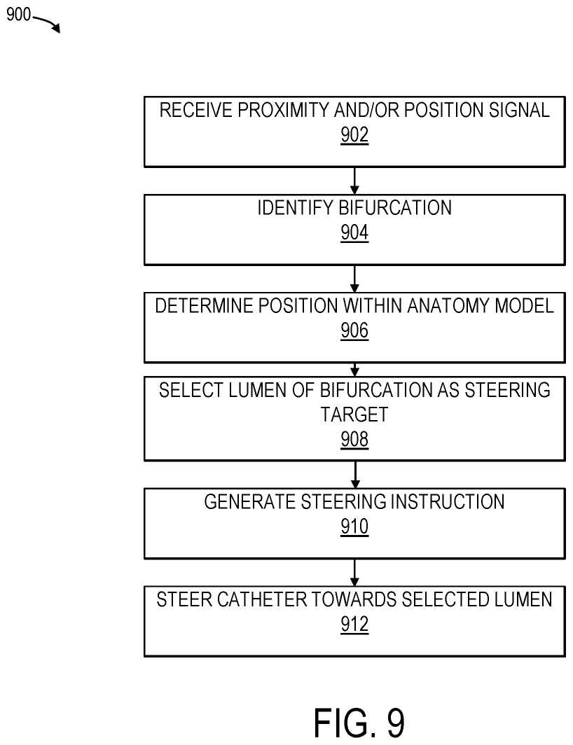

In some cases, multiple features are identified in the output from the ML model. For example, the branching of a pathway into at least two possible forward lumens can be identified by the feature identification model. Accordingly, operation 814 may include selecting one feature as the steering target. Selection of a particular feature of the multiple features may be based on an anatomical model of the patient as discussed further herein and in FIG. 9 below.

At operation 816, a position signal is received from a position sensor in the distal tip of the catheter. The position signal may provide information an orientation (e.g., a roll) of the catheter as well as information about movement of the catheter in the distal or proximal direction. At operation 818, a determination may be made as to whether the distal tip of the catheter is oriented towards the steering target. The orientation of the distal tip may also, or alternatively, be determined based on the proximity data. Where the orientation of the distal tip is not directed towards the steering target, a difference between the current orientation of the distal tip an orientation that would be required to cause the distal tip to be oriented towards the steering target may be determined. The difference may include a direction and/or a magnitude.

At operation 820 steering instructions are generated that, when performed, cause the distal tip to be oriented towards the steering target. The steering instructions may be based on the difference between the current orientation and the targeted orientation determined in operation 818. For instance, the steering instructions may include both a direction for bending the distal tip and a magnitude that the distal tip should be bent. At operation 822, the distal end is automatically steered (e.g., bent) based on the steering instructions. Method 800 may then repeat with new proximity data as the catheter continues to advance through the patient.

FIG. 9 depicts another example method 900 for automatically steering a catheter based on proximity data. At operation 902, a proximity signal is received from a proximity sensor in a distal tip of the catheter. A position signal may also be received from a position sensor in the distal tip of the catheter.

At operation 904, a junction bifurcation into at least two lumens (e.g., a first lumen and a second lumen) is detected based on the proximity data. Such a bifurcation may be determined based on pattern recognition or feature identification using ML models as discussed above.

At operation 906, a position of the distal tip within an anatomy model of the patient is determined. The position of the distal tip within the anatomy model may be based on the position signal and an initial registration of the catheter position with the anatomy model. The anatomy model may also include an ultimate navigation target within the patient.

At operation 908, a lumen of the bifurcation (e.g., the first lumen or the second lumen) is identified as the steering target. In some examples, the center of the selected lumen is identified as the steering target. The selection of the lumen may be based on the anatomy model, the current position of the distal tip in the anatomy model, and the location of the navigation target in the anatomy model. For instance, the lumen that will guide the catheter from its current position to the navigation target is the lumen that is selected. At operation 910, a steering instruction is generated to steer the distal tip towards the steering target, and at operation 912, the distal tip is steered according to the steering instructions. Generation of the steering instruction may also account for current orientation of the distal tip, such as discussed above with respect to operations 816-818 of method 800.

While the present techniques are discussed primarily in the context of catheter navigation within airway passages, it should be understood that the disclosed techniques may also be useful in other types of airway management or clinical procedures. For example, the disclosed techniques may be used in conjunction with placement of other devices within the airway, secretion removal from an airway, arthroscopic surgery, bronchial visualization past the vocal cords (bronchoscopy), tube exchange, lung biopsy, nasal or nasotracheal intubation, blood-vessel catheterization procedures, etc. The disclosed catheters may also be used for or in conjunction with suctioning, drug delivery, ablation, or other treatments of visualized tissue and may also be used in conjunction with endoscopes, bougies, introducers, scopes, or probes. Further, the disclosed techniques may also be applied to navigation and/or patient visualization using other clinical techniques and/or instruments, such as patient catheterization techniques. By way of example, contemplated techniques include cystoscopy, cardiac catheterization, catheter ablation, catheter drug delivery, or catheter-based minimally invasive surgery.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A catheter navigation system comprising:
a catheter comprising a steerable distal tip including a proximity sensor; and
a catheter controller, coupled to the catheter, comprising:
a processor; and
memory storing instructions that, when executed by the processor, cause the catheter controller to perform operations comprising:
receiving, from the catheter, a proximity signal generated by the proximity sensor;
identifying a steering target based on the proximity signal; and
generating a steering signal to steer the steerable distal tip towards the steering target.

2. The catheter navigation system of claim 1, wherein the proximity sensor includes at least one of an infrared sensor, an acoustic sensor, or a temperature sensor.

3. The catheter navigation system of claim 1, wherein the proximity sensor includes at least one proximity receiver positioned at least partially around a circumference of the distal tip.

4. The catheter navigation system of claim 1, wherein identifying the steering signal is further based on an anatomical model generated by an external imaging modality.

5. The catheter navigation system of claim 1, wherein the steerable distal tip further includes a position sensor, and generating the steering signal is further based on orientation data received from the position sensor.

6. The catheter navigation system of claim 1, wherein identifying the steering target comprises:
providing the proximity signal as input into a trained machine-learning (ML) model; and
receiving, as output from the ML model, the steering target.

7. The catheter navigation system of claim 1, wherein the steering target is identified by identifying features of the proximity signal characteristic of a lumen and selecting a center of the lumen as the steering target.

8. A catheter navigation system comprising:
a catheter comprising a steerable distal tip including a proximity sensor; and
a catheter controller, coupled to the catheter, comprising:
a processor; and
memory storing instructions that, when executed by the processor, cause the catheter controller to perform operations comprising:
receiving, from the catheter, a proximity signal generated by the proximity sensor;
based on the proximity signal, determining a proximity of the steerable distal tip to a lumen wall of a lumen; and
based on the determined of the steerable distal tip to the lumen wall, generating a steering signal to steer the steerable distal tip away from the lumen wall and towards a center of the lumen.

9. The catheter navigation system of claim 8, wherein determining the proximity of the steerable distal tip to one or more lumen walls comprises:
determining a first distance of a first side of the distal tip to a first lumen wall; and
determining a second distance of a second side of the distal tip to a second lumen wall, wherein the second distance is greater than the first distance; and
wherein the steering signal is to steer the distal tip towards the second side of the distal tip.

10. The catheter navigation system of claim 8, wherein the operations further comprise, based on the determined proximity, identifying the center of the lumen.

11. The catheter navigation system of claim 8, wherein the steering signal is a first steering signal, and the operations further comprise:
based on the proximity signal, identifying a bifurcation of a first lumen and a second lumen of a patient;
based on an anatomy model of the patient, identifying the first lumen as providing a pathway to a navigation target in the anatomy model; and
generating a second steering signal to steer the distal tip into the first lumen.

12. The catheter navigation system of claim 8, wherein the proximity sensor comprises:
a proximity emitter; and
at least one at least one proximity receiver positioned at least partially around a circumference of the distal tip.

13. A method for navigating a catheter while the catheter is being advanced through a lumen, the method comprising:
receiving, at a catheter controller, a proximity signal from a proximity sensor in a steerable distal tip of the catheter, the proximity sensor being at least one of an infrared sensor, an acoustic sensor, or a temperature sensor;
providing the proximity signal to a trained machine-learning (ML) model;
receiving an output form the ML model in response to the proximity signal;
identifying a steering target based on the output from the ML model; and
automatically bending, by the catheter controller, the distal tip towards the steering target.

14. The method of claim 13, wherein the steering target is a central axis of the lumen.

15. The method of claim 13, wherein the lumen is a first lumen, the catheter is positioned at a bifurcation to a second lumen and third lumen, and the steering target is one of the second lumen or the third lumen.

16. A catheter navigation system comprising:
a catheter comprising a steerable distal tip including a proximity sensor; and
a catheter controller, coupled to the catheter, comprising:
a processor; and
memory storing instructions that, when executed by the processor, cause the catheter controller to perform operations comprising:
receiving, from the catheter, a proximity signal generated by the proximity sensor,
based on the proximity signal, detecting an anatomical structure in proximity to the distal tip; and
generating a steering signal to steer the steerable distal tip away from the detected anatomical structure.

17. The catheter navigation system of claim 16, wherein the anatomical structure is a lumen wall.

18. The catheter navigation system of claim 16, wherein the anatomical structure is tissue dividing two lumens at a junction.
